(19) United States
(12) Patent Application Publication
(10) Pub. No.: US 2017/0298092 A1
(43) Pub. Date: Oct. 19, 2017

(54) FINE FIBER WEB WITH CHEMICALLY FUNCTIONAL SPECIES

(71) Applicants: Vishal Bansal, Lee's Summit, MO (US); Thomas D. Carr, Franklin, TN (US); Yogesh Ner, Spring Hill, TN (US); Kaiyi Liu, Spring Hill, TN (US); Stephen R. Kay, Austin, TX (US)

(72) Inventors: Vishal Bansal, Lee's Summit, MO (US); Thomas D. Carr, Franklin, TN (US); Yogesh Ner, Spring Hill, TN (US); Kaiyi Liu, Spring Hill, TN (US); Stephen R. Kay, Austin, TX (US)

(73) Assignee: CLARCOR Inc., Franklin, TN (US)

(21) Appl. No.: 15/490,586

(22) Filed: Apr. 18, 2017

Related U.S. Application Data
(60) Provisional application No. 62/324,784, filed on Apr. 19, 2016.

(51) Int. Cl. C07K 1/22; B01D 15/38; B01J 20/286; D01D 5/18; D01D 5/00; B01D 15/20; B32B 5/02; B01D 39/16

(52) U.S. Cl. CPC C07K 1/22; B32B 5/022; B01D 15/3804; B01D 39/163; D01D 5/18; D01D 5/0069; B01D 15/206; B01J 20/286; B01J 2220/82; B32B 2323/10; D10B 2505/04

(57) ABSTRACT

A functionalized fine fiber is provided. In an embodiment, the functionalized fine fiber is usable in chromatography. The functionalized fine fiber includes a matrix of fine fiber. The fine fibers preferably have an average diameter of less than 2 micron, and each fine fiber preferably has a length of at least 1 millimeter. The fine fibers carry and immobilize functional molecules.

### CROSS-REFERENCE TO RELATED PATENT APPLICATIONS

This patent application claims the benefit of U.S. Provisional Patent Application No. 62/324,784, filed Apr. 19, 2016, the entire teachings and disclosure of which are incorporated herein by reference thereto.

### FIELD OF THE INVENTION

This invention generally relates to a functionalized fine fiber, and more particularly, this invention relates to a functionalized fine fiber, usable in a variety of chromatography techniques.

### BACKGROUND OF THE INVENTION

Methods of and apparatuses for producing nanofibers are known by way of centrifugal spinning. Exemplary disclosures include U.S. Publication Nos. 2016/0083867, 2016/0069000, 2015/0013141, 2014/0339717, 2014/0217629, 2014/0217628, 2014/0159262, 2014/0042651, 2014/035179, 2014/0035178, 2014/0035177, 2012/0295021, and 2012/0294966 and U.S. Pat. Nos. 9,181,635; 8,778,240; 8,709,309; 8,647,541; and 8,647,540. These entire disclosures are incorporated in their entireties herein by reference. As such, centrifugal spinning, spinnerets, materials, and methods disclosed in these references are preferred for use in an embodiment of the present invention that provides for improvements and new uses for such centrifugal spinning systems.

### BRIEF SUMMARY OF THE INVENTION

The inventive aspects and embodiments discussed below in the following separate paragraphs of the summary may be used independently or in combination with each other.

In one aspect, a functionalized fine fiber is provided. In an embodiment, the functionalized fine fiber is usable in chromatography. The functionalized fine fiber includes a matrix of fine fiber. The fine fibers preferably have an average diameter of less than 2 micron, and each fine fiber preferably has a length of at least 1 millimeter. The fine fibers carry and immobilize functional molecules.

In certain embodiments, the functional molecules are ligands.

In specific embodiments, the ligands are antibodies specific to target proteins.

The fine fiber can be formed from at least one polymer selected from the group consisting of polytetrafluoroethylene, polyvinylidene fluoride, other fluoropolymers, polyamide, polyester, cellulose, polysulfone, polyethylene, polypropylene, polystyrene, poly(4-vinylpyridine).

In other embodiments, the functional molecules are at least one metal ion. The metal is preferably selected from the group consisting of: cobalt, nickel, copper, iron, zinc, and gallium.

In another embodiment, the functional molecules are hydrophobic groups.

Preferably, the hydrophobic groups include one or more of a phenyl group, an octyl group, and a butyl group.

The fine fiber can be formed into a fibrous web entanglement. In such embodiments, preferably the fibrous web entanglement has the following properties: a permeability of between 0.1 and 50 CFM/ft² at 0.5″ W.C.; a basis weight of between 1 grams/square meter and 100 grams/ square meter.

In an embodiment, the functionalized porous substrate can include a porous substrate layer supporting the fibrous web entanglement. In such embodiments, the porous substrate is preferably made from nonwoven scrims made from materials selected from the group consisting of polyester, polypropylene, polytetrafluorethylene, polyvinylidene fluoride, polyamides, and combinations thereof.

In another aspect, a method of separating chemical mixtures using the fine fibers is provided. A heterogeneous group of molecules is applied in solution, which includes target molecules. The target molecules are trapped via the functional molecules on the functionalized fine fiber, thereby generating a remainder solution. The remainder solution is removed from the functionalized fine fiber. The target molecules with a solvent are eluted from functionalized fine fiber, and the solvent with the target molecules is collected.

In a specific embodiment, the method is used in circumstances where the target molecule is a protein.

In the method, the step of eluting can be accomplished by changing at least one of salt concentrations, pH, charge and ionic strength directly or through a gradient to resolve the particles of interest.

In another aspect, a method of forming the functionalized fine fiber is provided. The fine fibers are formed by centrifugally expelling a liquid polymer that comprises at least one of polymer melt or polymer solution, through orifices in at least one spinneret while rotating the spinneret at a speed of at least 2500 rpms. The fiber diameter of the fine fibers is drawn down through centrifugal force and without the use of electrospinning forces to draw down the fiber diameter. The fine fibers from the liquid polymer melt or a polymer solution are entangled, and the polymer melt or polymer solution prior to forming by centrifugally spinning includes the functional molecules.

In still another aspect, a further method of forming the functionalized fine fiber is provided. The fine fibers are formed by centrifugally expelling a liquid polymer that comprises at least one of polymer melt or polymer solution through orifices in at least one spinneret while rotating the spinneret at a speed of at least 2500 rpms. A fiber diameter of the fine fibers is drawn down through centrifugal force and without the use of electrospinning forces to draw down the fiber diameter. The fine fibers from the liquid polymer are entangled, and the functional molecules are attached to the fibrous web entanglement after forming by centrifugally spinning by surface grafting, coating, or adhesion.

In still another aspect, the functionalized fine fiber can be contained in a fibrous web that has been laminated to a substrate to form a laminated material.

In certain embodiments, the substrate is polypropylene spunbond.

In further embodiments, the laminated material is pleated to form a filtration cartridge.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 depicts a schematic representation of a functionalized fine fiber according to an exemplary embodiment of the present invention;

FIG. 2 depicts a schematic representation (not to scale) of a manufacturing line for forming a fibrous web including the functionalized fine fiber of FIG. 1;

FIG. 3 depicts a schematic representation (not to scale) of the deposition chamber, including a spinneret, located on the manufacturing line depicted in FIG. 2;

FIG. 4 depicts an elution column packed with the fibrous web made on the manufacturing line depicted in FIG. 2;

FIG. 5 is a flowchart outlining the steps of performing affinity chromatography using the elution column depicted in FIG. 4;

FIG. 6 depicts a schematic representation (not to scale) of a functionalized fine fiber of FIG. 1 with a ligand bonding to a target molecule;

FIG. 7 depicts a schematic representation (not to scale) of a laminate material including the functionalized fine fiber of FIG. 1; and

FIG. 8 depicts a pleated chromatography column made from the laminate material depicted in FIG. 7.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Biological molecules can be separated through a variety of chromatography techniques. Affinity chromatography has a particular suitability for separating proteins, and the following discussion will primarily focus on protein separation and isolation. However, this discussion is provided by way of example only and not meant to limit the scope of the invention in any way.

The basis of affinity chromatography is a reversible interaction between a protein (or proteins) and a specific ligand. The ligand is bound to a nonreactive chromatography matrix, which is packed into an elution column. A solution containing the desired protein, among other molecules and compounds, flows through the matrix in the elution column. The desired protein will bind to the ligands while the other molecules and compounds in the solution will flow through the matrix without bonding or otherwise reacting. The solution and unbound molecules/compounds are then flushed from the elution tube, leaving the desired proteins bound to the ligands on the matrix. A second solution then flows through the elution column, which contains a competitive ligand or changes the pH, ionic strength, or polarity of reaction environment. Thus, the interaction between the ligand and the desired protein is no longer energetically favorable, and the desired protein will release from the ligand into the second solution, allowing the desired protein to be collected.

Conventional affinity chromatography uses functionalized packed beds of chromatography beads as the matrix to which the ligands are bonded. The beads are typically made of polystyrene or agarose and are spherical in shape.

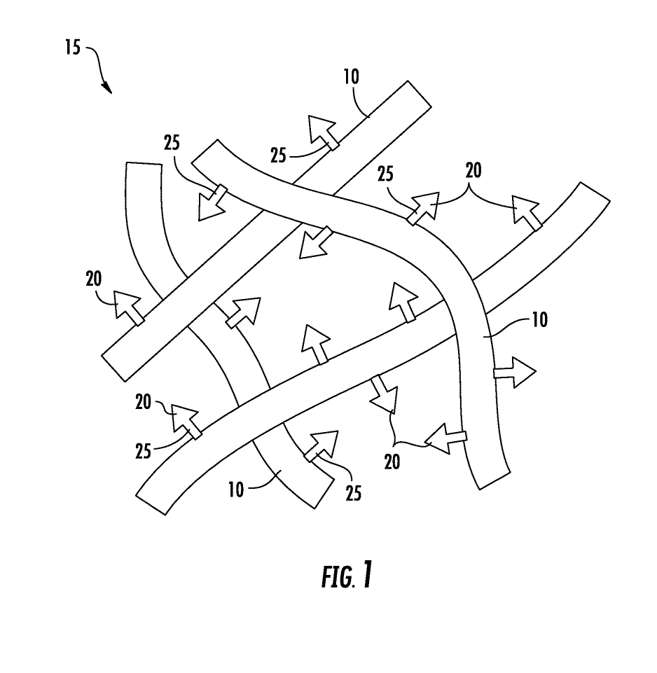

According to exemplary embodiments of the present invention, a nanofibrous web matrix comprised of functionalized fine fibers is provided. FIG. 1 depicts a schematic representation of the functionalized fine fibers 10 forming a chromatography matrix 15. The fine fibers 10 are functionalized to contain a plurality of functional molecules, which are depicted as ligands 20. The chromatography matrix 15 made from the functionalized fine fibers 10 advantageously provides more surface area for achieving high functionalization density and for the interaction of functional molecules with the target molecules in a solution. For instance, for a polymer with a specific gravity of 1-50%, a reduction in the fiber diameter will double the surface area, giving double the area for target molecule binding and improving capture efficiency and binding capacity. Thus, the increased surface area will also allow for faster solution (i.e., solution containing the target molecules) flow and higher productivity.

In some embodiments, the ligands 20 may be spaced from the fine fibers 10 using spacer arms 25. The spacer arms 25 are preferably molecules having a carbon backbone that is between 2 and 10 carbon atoms long. The spacer arms 25 move the ligand 20 away from the matrix 15 so that the desired protein has room to access the binding sites on the ligand 20. Suitable spacer arms include 1,6 diaminohexane, 6 amino hexonic acid, 1,4 bis (2,3 epoxypropoxy) butane, among others.

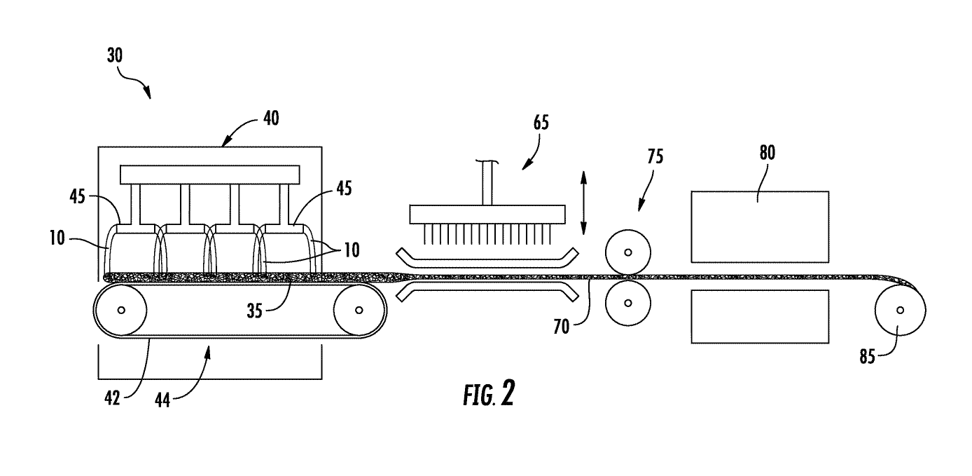

FIG. 2 depicts an exemplary embodiment of a manufacturing line 30 for creating the fine fibers 10. The fine fibers 10 are deposited as a loose batt 35 in a fiber deposition chamber 40. The fine fibers 10 are preferably produced via centrifugal spinning (herein referred to as “Forcespinning®”) and deposited on a moving substrate 42. The moving substrate 42 can be incorporated into the loose batt 35 of fine fibers 10, such as with a scrim material (i.e., a porous substrate), or the moving substrate can be separate from the loose batt 35 of fine fibers 10, such as a conveyor system 44 (as depicted in FIG. 1).

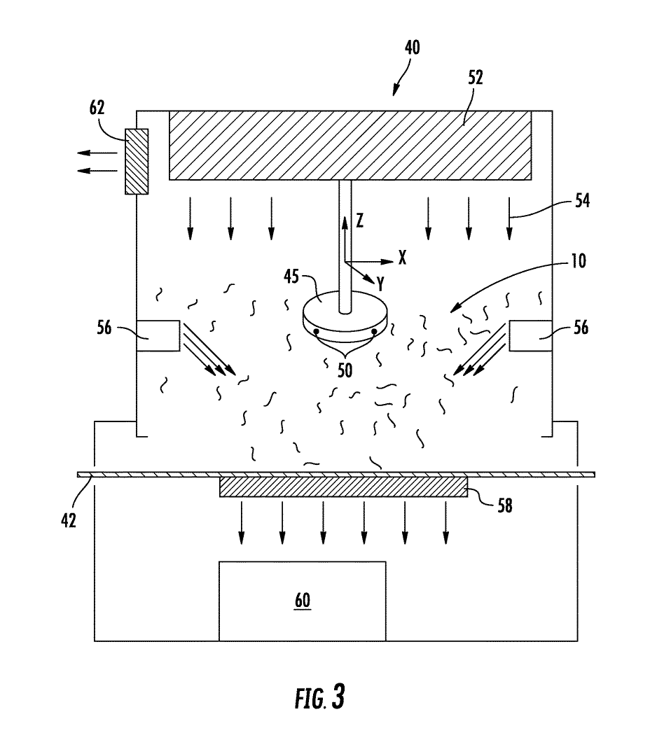

FIG. 3 depicts a more detailed schematic view of a section of the fiber deposition chamber 40. As depicted in FIGS. 2 and 3, the deposition chamber 40 is a Forcespinning® chamber. Forcespinning® involves centrifugally expelling a liquid polymer (i.e., at least one of a polymer melt or polymer solution) through orifices in at least one spinneret 45 while rotating the spinneret 45 at a speed of at least 2500 rpms. This centrifugal action results in the drawing down of the fiber diameter of the fine fibers. It should be noted that the Forcespinning® process does not use electrospinning forces to draw down the diameter of the fine fibers 10.

The deposition chamber 40 of FIG. 3 depicts a single spinneret 45, but more spinnerets 45 can be included in the deposition chamber 40, such as shown in FIG. 1, depending on the amount of fine fibers 10 needed. The spinnerets 45 typically are capable of moving in the X, Y, and Z planes to provide a range of coverage options for producing the loose batt 35. Each spinneret 45 features a plurality of orifices 50 through which the fine fibers 10 are expelled. The orifices 50 can each be connected to the same reservoir of polymer melt, polymer solution, or liquid adhesive, or each orifice 50 can be connected to a different reservoir of polymer melt, polymer solution, or liquid adhesive. Moreover, in embodiments with multiple spinnerets 45, each spinneret 45 can expel a different polymer melt, polymer solution, or liquid adhesive. During fine fiber deposition, the spinnerets 45 will rotate at least at 2500 rpms. More typically, the spinnerets 45 will rotate at least at 5000 rpms.

Using the spinnerets 45, the fine fibers 10 can be created using, for example, a solution spinning method or a melt spinning method. A polymer melt can be formed, for example, by melting a polymer or a polymer solution may be formed by dissolving a polymer in a solvent. Polymer melts and/or polymer solutions as used herein also refers to the material formed from heating the polymer to a temperature below the melting point and then dissolving the polymer in a solvent, i.e., creating a “polymer melt solution.” The polymer solution may further be designed to achieve a desired viscosity, or a surfactant may be added to improve flow, or a plasticizer may be added to soften a rigid fiber, or an ionic conductor may be added to improve conductivity. The polymer melt can additionally contain polymer additives, such as antioxidant or colorants.

Preferably, the ligand precursors and spacer arm precursors (if included) are added to the polymer solution prior to spinning the fibers. In this way, the fine fibers 10 will be functionalized during the spinning process. Thus, when the fibrous web is created from the fine fibers 10, the fibrous web will also be functionalized and ready for use in affinity chromatography. The ligand precursors react with the spacer arm or matrix to form the ligand. The spacer arm precursors react with the matrix to produce the spacer arm.

Several optional features of the deposition chamber 40 are depicted in FIG. 3. Generally, the fine fibers 10 are preferably continuous fibers (though the fine fibers 10 are depicted schematically as short fibers in FIG. 3). The fine fibers 10 can be encouraged downwardly to collect on the moving substrate 42 through a variety of mechanisms that can work independently or in conjunction with each other. For example, in some embodiments, a gas flow system 52 can be provided to induce a downward gas flow, depicted with arrows 54. The gas flow system 52 can also include lateral gas flow jets 56 that can be controlled to direct gas flow in different directions within the deposition chamber 40. Additionally, in some embodiments, formation of the fine fibers 10 will induce an electrostatic charge, either positive or negative, in the fiber. This electrostatic charge is not used to draw the fiber to the desired thickness such as in electrospinning. Nevertheless, an electrostatic plate 58 can be used to attract the charged fibers 10 downwardly to the moving substrate 42. Thus, as can be seen in FIG. 3, the electrostatic plate 58 is located below the moving substrate 42. Furthermore, in some embodiments, a vacuum system 60 is provided at the bottom of the deposition chamber 40 to further encourage the fine fibers 10 to collect on the moving substrate 42. Still further, in some embodiments, an outlet fan 62 is provided to evacuate any gasses that may develop, such as might develop as the result of solvent evaporation or material gasification, during the Forcespinning® process.

In other embodiments, the fine fiber 10 can be deposited using a different method than Forcespinning® or in conjunction with Forcespinning®. For example, in one embodiment, the fine fiber 10 can be produced via electrospinning.

The fine fiber strands 10 that are incorporated into the loose batt 35 have a length greater than 1 millimeter and an average diameter of less than 2 micron. More preferably, the fine fiber strands 10 have a length greater than 10 cm and an average diameter less than 2 micron, and most preferably, the fine fiber strands 10 have a length greater than 1 meter (i.e., continuous strands).

Returning to FIG. 2, the loose batt 35 of fine fibers 10 is transported out of the deposition chamber 40 on the moving substrate 42. The Forcespinning® process may produce enough fiber entanglement by itself that further entanglement is unnecessary. However, as depicted in FIG. 2, the loose batt 35 is transported to a needlepunching machine 65 to increase the amount of entanglement of the fine fibers 10. If a scrim or porous substrate is utilized, the needlepunching machine 65 can punch the fine fibers 10 into the scrim or porous substrate. Once the fibers are sufficiently entangled, either through Forcespinning® alone or through an entanglement process, such as needlepunching, the fine fibers 10 form a fibrous web 70.

Optionally, the fibrous web 70 can be further processed to enhance the bonding of the fibers or to increase the density of the media. As depicted in FIG. 2, the fibrous web 70 travels through calendaring rolls 75. Multiple sets of calendaring rolls can be utilized, and the calendaring rolls can be heated. Also, as depicted in FIG. 2, the fibrous web 70 travels through an oven 80, which can soften the fine fibers 10 such that the fine fibers 10 thermally bond to each other. At the end of the manufacturing line 30, the fibrous web 70 is taken up in a roll 85 for storage or transportation for further processing.

Preferably, the fibrous web 70 is made from one or more polymeric materials. Suitable polymers for the fine fiber 10 include polytetrafluoroethylene, polyvinylidene fluoride, other fluoropolymers, polyamide, polyester, cellulose, polysulfone, polyethylene, polypropylene, polystyrene, and poly(4-vinylpyridine).

Properties of a fibrous web 70 made according to the above-described method will typically be as follows. The air permeability of the fibrous web 70 will be between 0.1 and 50 CFM/ft² at 0.5″ W.C. (cubic feet per minute, per square foot, at half-inch water column). Additionally, the basis weight will be between 1 g/m² (grams per meter squared) and 100 g/m².

If the fine fibers 10 are not functionalized during the spinning process, the fibrous web 70 has to be activated in order to bind ligands 20 to the fine fibers 10. Suitable means of activating the fibrous web 70 include surface grafting, coating, spraying, and adhesion. Surface grafting can be done in the “graft to” or “graft from” approaches. Chemical or radiation processes (e.g., plasma) can be used to drive the grafting reaction. Once the fibrous web 70 is activated the optional spacer arms 25 can be added to the activated fibrous web 70. After activation of the fibrous web (or after attachment of the spacer arm 25 is utilized), the ligands 20 are added. Suitable ligands include antibodies specific to target proteins.

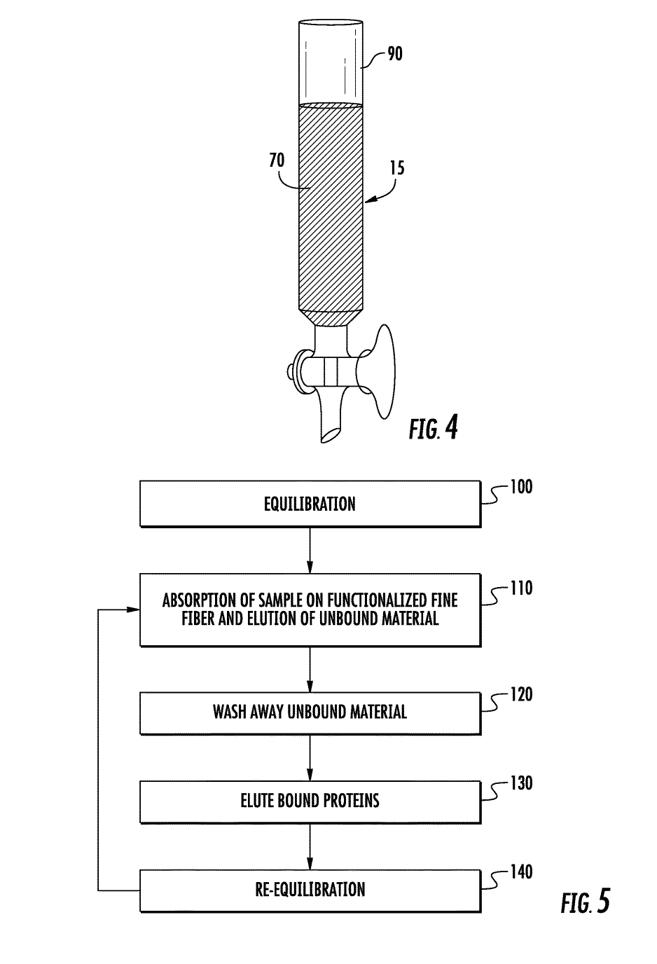

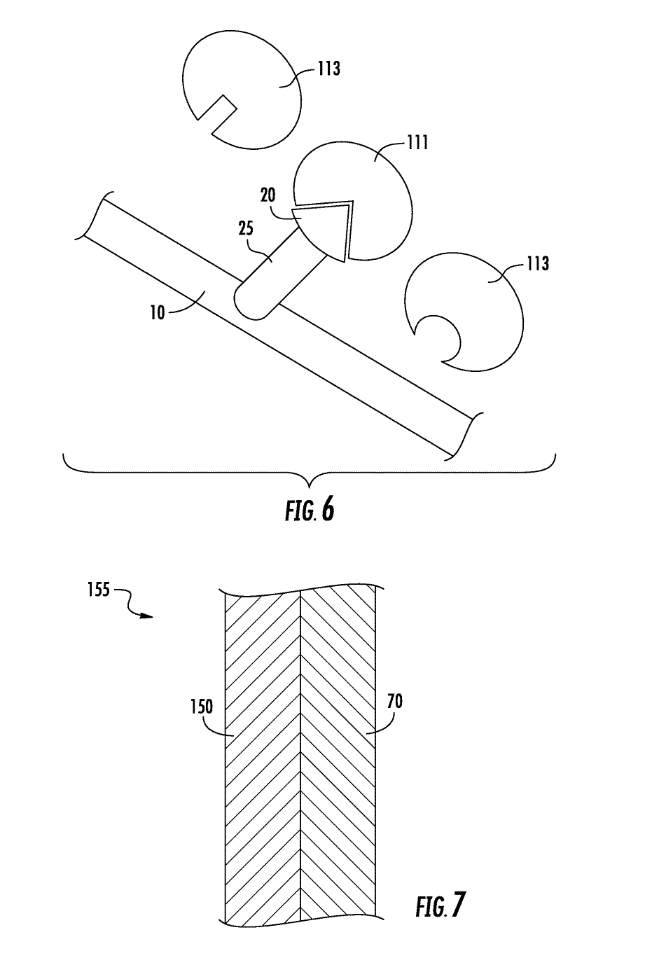

Once functionalized with the ligands 20, the fibrous web 70 is packed into an elution column 90 as shown in FIG. 4. Thereafter, a separation can be performed. FIG. 5 depicts the steps of a bioseparation according to the affinity chromatography technique. The first step 100 involves equilibrating the fibrous web 70 (which serves as the matrix 15) of the elution column 90. A sample containing a heterogeneous group of molecules in solution, including the target molecule, is poured into the elution column 90. In the second step, the target molecules are absorbed on the functionalized fine fibers 10 of the fibrous web 70 via the ligands 20. Binding occurs by intermolecular forces, such as ionic bonds, hydrogen bonds and Van der Waals forces. FIG. 6 is a schematic depiction of a target molecule 111 binding to a ligand 20. Also depicted are two other unbound molecules 113, which do not display an affinity for the ligand 20 and, therefore, do not bind to the ligand 20. Thus, the other unbound molecules remain in the solution, which is eluted from the elution column 90.

In a third step 120, any remaining unbound molecules are washed away with a buffer solution. In a fourth step 130, the target molecules are eluted by changing the salt concentration, pH, pI (isoelectric point), charge and/or ionic strength directly or through a gradient of the elution column. This unbinds the target molecule from the ligand so that the target molecule can be eluted and collected. In a final step 140, the elution column is re-equilibrated so that additional sample solution can flow through the elution column.

Advantageously, the fibrous web 70 has a much higher surface area and a wider pore size distribution than conventional chromatography beads. Accordingly, the fibrous web 70 has more area for ligands 20 to bind target molecules.

While the foregoing description primarily focused on protein bioseparation affinity chromatography, the disclosure applies broadly to other chromatography techniques. For instance, the functionalized fine fibers 10 can also be used in immobilized metal affinity chromatography (IMAC) (also known as metal chelate affinity chromatography (MCAC)). In IMAC, transition metal ions, such as zinc, copper, cobalt, nickel, iron, and gallium, can coordinate to the amino acids histidine, cystein, and tryptophan via electron donor groups on the amino acid side chains. The metal ion, i.e., functional molecule, is immobilized on the fine fibers 10. The metal ion is attached via a chelating group to the chromatographic matrix 15 (i.e., the nanofibrous web 70). Preferably, the metal ion is attached with a long hydrophilic spacer arm that ensures the chelating metal is fully accessible to all available binding sites on a protein.

Other chromatography techniques that the present disclosure can be applied to include ion chromatography, hydrophobic interaction chromatography, and reversed phase chromatography, among others. In each of these chromatography techniques, a functional molecule is used to attract and bind a specific target molecules among many molecules contained in a solution. Using the aforedescribed manufacturing methods, the functional molecule can be incorporated into a nanofibrous web, thereby providing an increase in the amount of surface area for the functional molecule to interact with the target molecule.

The functionalized fibers 10 are applicable to such fields as biopharmaceutical manufacturing, biofuel manufacturing, and waste water remediation, among others, in which separating molecules from a solution is desired.

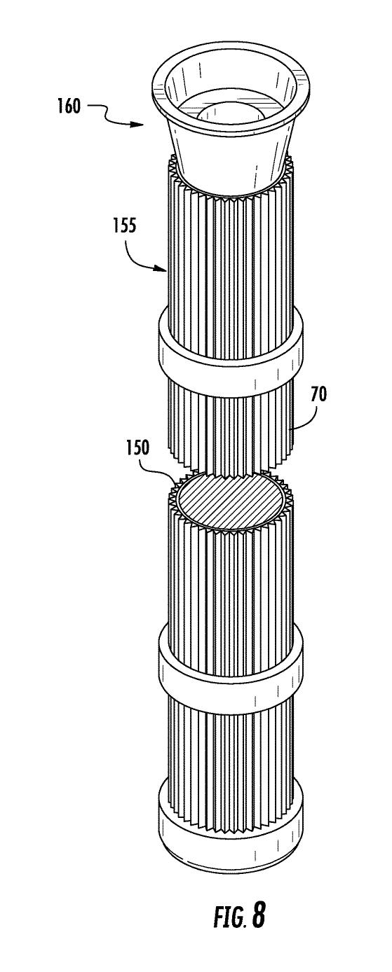

In another embodiment, fibrous web 70 can be laminated with a nonwoven substrate 150, such as polypropylene spunbond. FIG. 7 depicts an schematic representation of a laminated material 155. This laminated material 155 can then be pleated into filtration cartridges 160 as depicted in FIG. 8. The filtration cartridges 160 can be used in lieu of the traditional affinity chromatography packed columns.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A functionalized fine fiber, usable in chromatography, comprising:
a matrix of fine fiber, the fine fiber having an average diameter of less than 2 micron, each fine fiber having a length of at least 1 millimeter; and
functional molecules carried and immobilized by the fine fiber.

2. The functionalized fine fiber of claim 1, wherein the functional molecules are ligands.

3. The functionalized fine fiber of claim 2, wherein the ligands are selected from the group consisting of antibodies specific to target proteins.

4. The functionalized fine fiber of claim 1, further wherein the fine fiber is formed of at least one polymer selected from the group consisting of polytetrafluoroethylene, polyvinylidene fluoride, other fluoropolymers, polyamide, polyester, cellulose, polysulfone, polyethylene, polypropylene, polystyrene, and poly(4-vinylpyridine).

5. The functionalized fine fiber of claim 1, wherein the functional molecules comprise at least one metal ion, the metal of the at least one metal ion selected from the group consisting of: cobalt, nickel, copper, iron, zinc, and gallium.

6. The functionalized fine fiber of claim 1, wherein the functional molecules are hydrophobic groups.

7. The functionalized fine fiber of claim 6, wherein the hydrophobic groups include one or more of a phenyl group, an octyl group, and a butyl group.

8. The functionalized fine fiber of claim 1, wherein the fine fiber is contained in a fibrous web entanglement having:
a permeability of between 0.1 and 50 CFM/ft² at 0.5″ W.C.;
a basis weight of between 1 grams/square meter and 100 grams/square meter.

9. The functionalized fiber of claim 8, further comprising a porous substrate layer supporting the fibrous web entanglement, the porous substrate comprising a nonwoven scrim made from a material selected from the group consisting of polyester, polypropylene, polytetrafluoroethylene, polyvinylidene fluoride, polyamides, and combinations thereof

10. A method of separating chemical mixtures using the fine fibers as in claim 1, comprising:
applying a heterogeneous group of molecules in solution, including target molecules;
trapping the target molecules via the functional molecules on the functionalized fine fiber, thereby generating a remainder solution;
removing the remainder solution from the functionalized fine fiber;
eluting the target molecules with a solvent from functionalized fine fiber; and
collecting the solvent with the target molecules.

11. The method of claim 10, wherein the target molecule is a protein.

12. The method of claim 10, wherein said eluting comprises at least one of changing salt concentrations, pH, pI, charge and ionic strength directly or through a gradient to resolve the particles of interest.

13. A method of forming the functionalized fine fiber of claim 1, comprising:
forming the fine fibers by centrifugally expelling a liquid polymer that comprises at least one of polymer melt or polymer solution, through orifices in at least one spinneret while rotating the spinneret at a speed of at least 2500 rpms;
drawing down a fiber diameter of the fine fibers through centrifugal force without the use of electrospinning forces to draw down the fiber diameter; and
entangling the fine fibers from the liquid polymer melt or a polymer solution,
wherein the polymer melt or polymer solution prior to forming the fine fibers comprises the functional molecules.

14. A method of forming the functionalized fine fiber of claim 1, comprising:
forming the fine fibers by centrifugally expelling a liquid polymer that comprises at least one of polymer melt or polymer solution through orifices in at least one spinneret while rotating the spinneret at a speed of at least 2500 rpms;
drawing down a fiber diameter of the fine fibers through centrifugal force without the use of electrospinning forces to draw down the fiber diameter;
entangling the fine fibers from the liquid polymer, and
attaching the functional molecules to the fibrous web entanglement after forming the fine fibers by surface grafting, coating, spraying, or adhesion.

15. The functionalized fine fiber of claim 1, wherein the functionalized fine fiber is contained in a fibrous web that has been laminated to a substrate to form a laminated material.

16. The functionalized fine fiber of claim 15, wherein the substrate is polypropylene spunbond.

17. The functionalized fine fiber of claim 15, wherein the laminated material is pleated to form a filtration cartridge.
